(19) United States
(12) Patent Application Publication
(10) Pub. No.: US 2025/0308695 A1
(43) Pub. Date: Oct. 2, 2025

(54) CLINICALLY DRIVEN METHOD FOR THE TREATMENT OF INFRAPOPLITEAL LESIONS IN SUBJECTS WITH CRITICAL LIMB ISCHEMIA

(71) Applicant: Micro Medical Solutions, Inc., Woburn, MA (US)

(72) Inventors: Rita Jacob, Eugene, OR (US); Bret Wiechmann, Gainesville, FL (US)

(21) Appl. No.: 19/092,164

(22) Filed: Mar. 27, 2025

Related U.S. Application Data
(60) Provisional application No. 63/570,437, filed on Mar. 27, 2024.

(51) Int. Cl. G16H 50/20; G16H 20/00

(52) U.S. Cl. CPC G16H 50/20; G16H 20/00

(57) ABSTRACT

A method for treatment of critical limb ischemia that includes determining if a patient has pain, establishing if the pain is an ulcer via testing, treating inflow (if existent), determining if a targeted vessel has blood flow, determining if a targeted lesion has blood flow, establishing if outflow is a cause of the occlusion, treating outflow (if existent), performing cardiovascular-death targeted lesion reintervention when applicable, performing a targeted lesion blood flow analytical angiography when applicable, and establishing if an occlusion is narrowed, establishing if an occlusion is blocked and treating each outcome.

### CROSS REFERENCE TO RELATED APPLICATION

This application claims the benefit of and takes priority from U.S. Provisional Application Ser. No. 63/570,437 filed on Mar. 27, 2024, the contents of which are herein incorporated by reference.

### BACKGROUND OF THE INVENTION

### Field of the Invention

This disclosure relates generally to a method for treatment of infrapopliteal lesions. Specifically, this disclosure relates to a method for treatment of infrapopliteal lesions in subjects with critical limb ischemia (CLI).

### Description of the Related Art

Atherosclerosis is the buildup of plaque on the inner lining of arteries. Causes include high cholesterol, high triglyceride levels, high blood pressure, smoking, diabetes, obesity, lack of physical activity, and high saturated fat consumption. As a result, index procedures are sought to revascularize the subject's arteries. However, index procedure often fails to provide a long-term resolution. As a result, a method for treatment of infrapopliteal lesions in subjects with critical limb ischemia was developed as a possible long-term alternative.

Current procedures to resolve arterial plaque buildup and implement revascularization can be problematic as very often these procedures do not provide revascularization that persists for more than six months. Additionally, these procedures can at times prove ineffective to the point where major amputations occur following initial index procedures.

Furthermore, arterial plaque may narrow the inner walls of an artery, at times consequentially causing target lesion occlusion and irregular blood flow in arteries and veins. Additionally, because index procedures often fail to provide lasting solutions for plaque occlusions, sizes of ischemic leg/foot ulcers are not reduced, causing further complications.

### SUMMARY OF THE INVENTION

Featured is a method for treatment of infrapopliteal lesions in subjects with critical limb ischemia that provides lasting arterial revascularization. Featured is a method for treatment of infrapopliteal lesions in subjects with critical limb ischemia that prevents Major Adverse Limb Events (MALE) such as above ankle amputations. Featured herein in one embodiment is a method for the treatment of infrapopliteal lesions that may occur in subjects with critical limb ischemia wherein the method may be utilized to reduces the size of leg and foot ulcers that often accompany critical ischemic.

Herein introduced is a method for treatment of infrapopliteal lesions in subjects with critical limb ischemia that provides lasting arterial vascularization, prevents limb amputations, and reduces sizes of ischemic leg/foot ulcers by providing steps to either treat inflow, outflow, target vessels, target lesions, or perform target lesion reintervention (TLR).

Additionally, the following disclosure involves a method for treatment of infrapopliteal lesions in subjects with critical limb ischemia that provides clear guidance and methodology for physicians, technicians and the like, unlike traditional infrapopliteal lesion treatments. In one embodiment, this guidance may be introduced as a decision tree detailing steps for procedural advancements.

The present disclosure provides a progressive method of treating infrapopliteal lesions in subjects with critical limb ischemia, including the following steps: determining if a patient has pain, establishing if the pain is an ulcer via testing, treating inflow (if existent), determining if a targeted vessel has blood flow, determining if a targeted lesion has blood flow, establishing if outflow is a cause of the occlusion, treating outflow (if existent), performing cardiovascular-death targeted lesion reintervention when applicable, performing a targeted lesion blood flow analytical angiography when applicable, establishing if an occlusion is narrowed, and establishing if an occlusion is blocked, and treating each outcome.

### BRIEF DESCRIPTION OF THE DRAWINGS

Advantages of the present device will be apparent from the following brief description of exemplary embodiments thereof; which description should be considered in conjunction with the accompanying drawings. Having thus described the invention in general terms, reference will now be made to the accompanying drawings, which are not necessarily drawn to scale, and wherein:

FIG. 1 illustrates one embodiment of the decision tree and accompanying method of determining occlusion causes (including femoral-popliteal disease and tibial/peroneal arterial occlusion disease) and selecting treatment procedures to properly address and correspond with the causes of the occlusion(s).

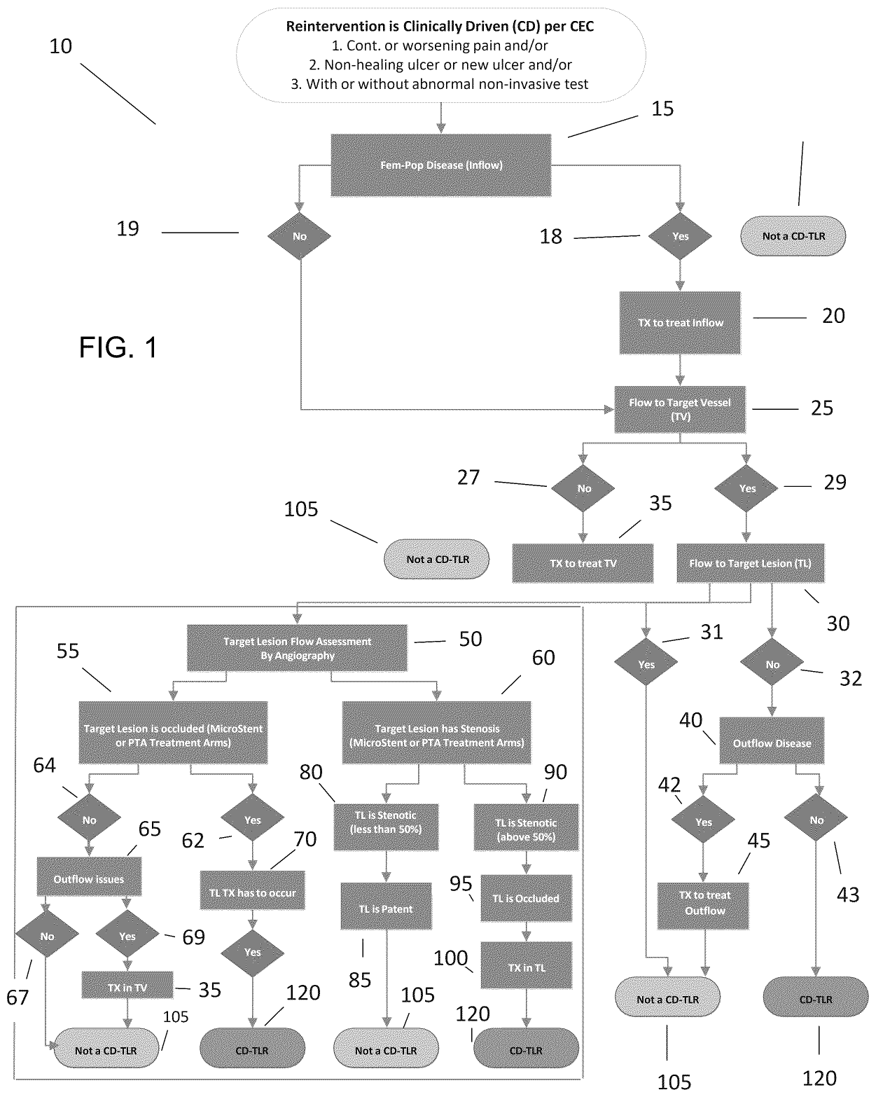

### DETAILED DESCRIPTION OF THE SEVERAL EMBODIMENTS

The present disclosure provides an interactive method of treating infrapopliteal lesions in subjects with critical limb ischemia. In one embodiment, a clinically driven reintervention may be utilized, wherein procedures are performed to address problems that occur as a result of the index procedure or the advancement of the disease pathology. These may also be associated with complaints of continued or worsening leg pain, progressing non-healing ulcers, or new ulcer formation, with or without the presence of an abnormal non-invasive test, including following a set of progressive steps.

In one embodiment, a set of progressive steps may include the following: determining if a patient has pain, establishing if the pain is an ulcer via testing, treating inflow 20 (if existent), determining if a targeted vessel has blood flow 25, determining if a targeted lesion has blood flow 30, establishing if outflow is a cause of the occlusion 40, treating outflow 45 (if existent), performing cardiovascular-death targeted lesion reintervention 120 when applicable, performing a targeted lesion blood flow analytical angiography 50 when applicable, and establishing if an occlusion is narrowed 80, establishing if an occlusion is blocked 90 and treating each outcome.

In the present disclosure, in one embodiment, the associated pain is preferably continued or worsening pain. Furthermore, when the continued or worsening pain is not existent, in another embodiment, a non-healing or new ulcer may satisfy as the starting point of the method. Moreover, in another embodiment, the starting point of the present method may begin with or without an abnormal, non-invasive procedure/test.

Following the initial starting point process, as described above, the method proceeds with determining if a patient has inflow 15. Inflow refers to the vascular pathway above the target lesion that could be the cause of occlusion above or at the target lesion. Inflow is a term that refers to occlusion(s) in the iliac, femoral, popliteal and tibioperoneal trunk that inhibits blood flow to a target vessel and lesion.

In one embodiment, inflow is the cause of the occlusion 18. Inflow 15 is not a cardiovascular death, but does require a targeted lesion reintervention 105 to be performed. When femoral popliteal disease 15 occurs, treatment 20 is needed to treat the inflow 15 before proceeding to a targeted vessel. Treatments include angioplasties and/or stent placements. In one embodiment, inflow is not the cause of the occlusion 19. When this occurs, treatment for inflow can be bypassed in order to proceed toward determining if a patient has blood flow to a targeted vessel 25.

In the present disclosure, after either treating inflow in the patient 20 or not treating inflow 19, a determination is to be made as to whether the patient has blood flow to the targeted vessel 25. If the patient does not have blood flood to the targeted vessel 27, it is not a cardiovascular death, but does require a targeted lesion reintervention 105 to be performed. As a result, treating the occlusion 35 is the next step in the method. Possible Treatment methods and procedures may consist of interventional procedures, surgical procedures or endovascular procedures (PTA, atherectomy, stenting), in order to restore patency or blood flow within the below the knee artery that was the target of the treatment at the index procedure.

In one embodiment, the reintervention may be a target vessel reintervention that is designed not to involve the target lesion, nor other vessels (i.e. Inflow and/or Outflow). When this occurs, it is not classified as a cardiovascular death. A cardiovascular death includes death resulting from an acute myocardial infarction (MI), sudden cardiac death, death due to heart failure (HF), death due to stroke, death due to cardiovascular (CV) procedures, death due to CV hemorrhage, and death due to other CV causes.

In one embodiment, when blood flow to the patient's targeted vessel is present 29, a user must determine if the patient has blood flow to the actual targeted lesion 30. In one embodiment, the patient may need target vessel reintervention and target lesion reintervention. These are defined as interventional procedures, surgical or endovascular (PTA, atherectomy, stenting), to restore patency or blood flow within the target limb that was treated at the index procedure.

Moreover, there are two types of target lesion reinterventions, major and minor. A major target lesion reintervention features the creation of a new surgical bypass, in some embodiments realized through the use of thrombectomy or thrombolysis (i.e., procedures done in the setting of lost secondary patency), or major surgical revision such as a jump graft or an interposition graft performed for critical occlusion of the target lesion. A minor target lesion reintervention is an endovascular procedure (PTA, atherectomy, stenting) without thrombectomy/thrombolysis, and minor surgical revisions (e.g. patch angioplasty) performed to prevent occlusion of the target lesion only.

Target lesion revascularization (TLR) can be defined as revascularization post-stenting within the stent or within the 5 mm borders adjacent to the stent. Ischemia-driven TLR (ID-TLR) was defined as revascularization of the target lesion based on clinical features of ischemia. Traditionally, target lesion revascularization (TLR) has been used in many of the interventional trials designed to test various pharmacology, while target vessel revascularization (TVR) has been used in device trials in which the effect of the device on the vessel beyond the target lesions also needs to be considered.

Traditionally, target lesion revascularization (TLR) has been used in many of the interventional trials designed to test various pharmacology, while target vessel revascularization (TVR) has been used in device trials in which the effect of the device on the vessel beyond the target lesions also needs to be considered.

In one embodiment, the reintervention is both a Target Vessel Reintervention (TVR) and a Target Lesion Reintervention (TLR). However, when determining if there is blood flow to the target lesion 30, in the present disclosure, there are three possible outcomes. First, if there is blood flow 31, this is not a cardiovascular death, but does require a targeted lesion reintervention 105 to be performed. Second, if there is not blood flood to the targeted lesion 32, a user must determine if outflow is the cause of an occlusion 40. If yes 42, treatment is required to treat the outflow 45. This is not a cardiovascular death but requires target lesion reintervention 105.

However, if outflow is not the cause of the occlusion 43, then this is a cardiovascular death that requires a target lesion reintervention 120.

Third, if a determination cannot be made as to whether the patient has blood flow to the target lesion, a target lesion flow assessment via an angiography 50 must be performed to determine whether the target lesion is occluded 55 or has stenosis 60.

In one embodiment, following the angiography, a determination is made that the target lesion is occluded 62. Consequentially, a MicroStent® Peripheral Vascular Stent System for the treatment of infrapopliteal lesions 70 in subjects with critical limb ischemia (CLI) is utilized for treatment of the occlusion. The procedure consists of two arms; one arm treated with PTA plus the MicroStent® System and one arm treated with PTA alone. The user will adjudicate whether an event is related to the index procedure, the device or unrelated to the device or procedure.

If the patient was randomized to the MicroStent® arm [PTA (percutaneous transluminal angioplasty) plus MicroStent® deployment], the user will adjudicate whether the event is related to the MicroStent® or PTA. If not related to the MicroStent®, the user will indicate whether the event is related to the index procedure or unrelated to the device or the index procedure.

If the event was related to another device that was used in the target limb, the user will adjudicate whether the event was related to that. If the patient was randomized to the PTA arm of the study, the CEC will determine whether an event was related to the PTA at the index procedure. As a result, in one embodiment, cardiovascular death occurs and target lesion reintervention 120 is required.

In one embodiment, the target lesion is not occluded 64. Consequentially, a determination must be made regarding if outflow issues are the cause of the pain, ulcer, etc. If there are no outflow issues 67, this is not a cardiovascular death, but does require a targeted lesion reintervention 105 to be performed. If outflow issues are present 69, treatment in the target vessel 35 is required. Outflow issues 69 are not a cardiovascular death, but does require a targeted lesion reintervention 105 to be performed.

In another embodiment, following the angiography 50, the target lesion has stenosis 60. Two types of stenosis occurs, less than 50% 80 and above 50% 90. When stenosis is below 50% 80, the target lesion is patent 85, a cardiovascular death is not present, and a target lesion reintervention 105 is required. When stenosis is above 50% 90, the target lesion is occluded 95. Target lesion occlusion 95 is the absence of flow on color Doppler, and/or absence of flow on angiographic images (conventional or CT). When this occurs, treatment 100 must be performed. A cardiovascular death occurs and a target lesion reintervention 120 is required.

In some embodiments, the patient has a bailout, and the user will adjudicate whether it was related to PTA. If a patient had a bailout and subsequently has a target limb reintervention, the user will determine whether the reintervention was related to bailout treatment (i.e. a DES was placed in the target lesion due to a dissection caused by PTA). If the patient has a target limb reintervention, the user will adjudicate whether the target limb reintervention is related to the MicroStent®, PTA, another implanted device or none of these in the target limb or the index procedure or unrelated to device or procedure.

The disclosed method is to be utilized to counteract adverse events, device related adverse events, and device deficiency. An adverse event is any untoward medical occurrence in a patient or clinical investigation subject whether or not it is considered to be device or procedure related. In addition, the definition of AE applies to any untoward medical event with an onset post study procedure or to any underlying diseases, present at baseline that exacerbate in severity post study procedure. Therefore, an underlying disease that was present at the time of enrollment is not reported as an AE, but any increase in the severity of the underlying disease is to be reported as an AE.

Serious adverse events (SAE) may be defined as any adverse event that meets at least one of the following: led to death; led to serious deterioration in the health of the subject that: results in life-threatening illness or injury, results in a permanent impairment of a body structure or a body function, requires inpatient hospitalization (>24 hrs) or prolongation of existing hospitalization, results in medical or surgical intervention to prevent permanent impairment to a body structure, function; led to fetal distress, fetal death, or a congenital abnormality or birth defect.

Device related adverse events may be defined as events directly attributable to the device itself, as determined by a Principal Investigator. A serious device-related adverse event is an adverse device effect that has resulted in any of the consequences characteristic of a serious adverse event. A device deficiency is an inadequacy of a medical device related to its identity, quality, durability, reliability, safety or performance, such as malfunction, misuse or use error and inadequate labeling. An unanticipated adverse device effect (UADE) is any serious adverse device effect which was not previously identified in nature, severity, or degree of incidence in the applicable product information (Clinical Investigational Plan, IFU, etc.). A procedure-related adverse event is an event that occurs from the procedure, irrespective of the device, as determined by a Principal Investigator.

The method of treatment of infrapopliteal lesions in subjects with critical limb ischemia 10 provides lasting arterial revascularization by determining the source of the occlusion. The method for treatment of infrapopliteal lesions in subjects with critical limb ischemia 10 prevents Major Adverse Limb Events (MALE), such as above ankle amputations, by associating the source of the occlusion with a proposed, corresponding treatment. The method for treatment of infrapopliteal lesions in subjects with critical limb ischemia 10 reduces sizes of ischemic leg/foot ulcers by providing accurate diagnostic analysis combined with appropriate counteractive treatment.

What is claimed is:

1. A method for treatment of infrapopliteal lesions comprising the steps of:
establishing whether a patient has a continuous pain, a worsening pain, a non-healing ulcer, or a new ulcer;
performing a diagnostic test further comprising;
establishing whether the patient has a first occlusion in an iliac, a femoral, a popliteal, or a tibioperoneal trunk that inhibits blood flow to a targeted vessel and a targeted lesion;
treating the first occlusion when present;
establishing whether the patient has blood flow in the targeted vessel;
treating the targeted vessel when a patient does not have blood flow;
establishing whether the patient has blood flow to the targeted lesion;
determining whether the patient has a second occlusion at or distal to the targeted lesion or into a plantar arteries of a foot;
wherein the second occlusion in a tibial and a peroneal arteries associates with the targeted lesion;
treating the second occlusion at or distal to the targeted lesion or into the plantar arteries of the foot when present;
wherein the second occlusion in the tibial and the peroneal arteries associates with the targeted lesion;
performing a cardiovascular death target lesion reintervention when a cause of the second occlusion is not caused by blockage at or distal to the targeted lesion or into the plantar arteries of the foot;
performing an angiography when it cannot be determined whether the patient has blood flow to the targeted lesion;
determining whether the targeted lesion is occluded;
determining whether the targeted lesion has stenosis;
treating the targeted lesion via the cardiovascular death target lesion reintervention when the targeted lesion is occluded;
treating the targeted vessel if outflow is the cause of the second occlusion;
analyzing the targeted lesion;
determining if the stenosis is above or below fifty percent in the targeted lesion; and
treating the stenosis with the cardiovascular death target lesion reintervention when the targeted lesion is occluded above fifty percent.

2. A method for treatment of critical limb ischemia comprising the steps of:
determining if a patient has pain;
establishing if the pain is an ulcer via testing;
treating inflow, when inflow is existent;
determining if a targeted vessel has blood flow;
determining if a targeted lesion has blood flow;
establishing if outflow is a cause of the occlusion;
treating outflow, when outflow is existent;
performing cardiovascular-death targeted lesion reintervention when applicable;
performing a targeted lesion blood flow analytical angiography when applicable; and
establishing if an occlusion is narrowed, establishing if an occlusion is blocked and treating each outcome.
